(19) United States
(12) Patent Application Publication
(10) Pub. No.: US 2020/0094014 A1
(43) Pub. Date: Mar. 26, 2020

(54) WAKEFULNESS MAINTAINING APPARATUS, WAKEFULNESS MAINTAINING METHOD, AND NON-TRANSITORY COMPUTER READABLE STORAGE MEDIUM

(71) Applicant: DENSO CORPORATION, Kariya-city (JP)

(72) Inventor: Masaru KAKIZAKI, Kariya-city (JP)

(21) Appl. No.: 16/571,253

(22) Filed: Sep. 16, 2019

(30) Foreign Application Priority Data
Sep. 20, 2018 (JP) 2018-175756

(51) Int. Cl. A61M 21/00; B60H 1/00

(52) U.S. Cl. CPC A61M 21/00; B60H 1/00742; A61M 2021/0066; A61M 2205/3606; A61M 2205/362; A61M 2205/3372; A61M 2205/3313; A61M 2021/0083

(57) ABSTRACT

A wakefulness maintaining apparatus includes: an awakening stimulation controller that controls an awakening stimulation device to generate a cold air wind for maintaining a wakeful state of a target person; and a characteristic feature specification unit that specifies a characteristic feature of the target person. The awakening stimulation controller changes an air wind stimulation condition based on the characteristic feature of the target person specified by the characteristic feature specification unit. The air wind stimulation condition includes at least one of a region of the target person which the cold air wind generated from the awakening stimulation device hits, an air volume of the cold air wind, a temperature of the cold air wind, and an air blowing period of the cold air wind.

### CROSS REFERENCE TO RELATED APPLICATION

The present application claims the benefit of priority from Japanese Patent Application No. 2018-175756 filed on Sep. 20, 2018. The entire disclosure of the above application is incorporated herein by reference.

### TECHNICAL FIELD

The present disclosure relates to a wakefulness maintaining apparatus, a wakefulness maintaining method, and a non-transitory computer readable storage medium each for maintaining a wakeful state of a target person.

### BACKGROUND

Conventionally, a technique which gives a stimulus to a driver so as to maintain a wakeful state of the driver has been known. For example, a technique alternately gives, to left and right hands and a neck of a driver, a stimulus for maintaining wakefulness of the driver using low-temperature air blasts from left and right vents of an air conditioning system.

### SUMMARY

According to an example embodiment, a wakefulness maintaining apparatus includes: an awakening stimulation controller that controls an awakening stimulation device to generate a cold air wind for maintaining a wakeful state of a target person; and a characteristic feature specification unit that specifies a characteristic feature of the target person. The awakening stimulation controller changes an air wind stimulation condition based on the characteristic feature of the target person specified by the characteristic feature specification unit. The air wind stimulation condition includes at least one of a region of the target person which the cold air wind generated from the awakening stimulation device hits, an air volume of the cold air wind, a temperature of the cold air wind, and an air blowing period of the cold air wind.

### BRIEF DESCRIPTION OF THE DRAWINGS

The above and other objects, features and advantages of the present disclosure will become more apparent from the following detailed description made with reference to the accompanying drawings. In the drawings:

FIG. 1 is a view showing an example of a schematic configuration of a wakefulness maintaining system;

FIG. 2 is a view showing an example of a schematic configuration of an air conditioning device;

FIG. 3 is a view showing an example of a schematic configuration of a HCU;

FIG. 4 is a view for illustrating an example of tables associated in advance with different characteristic features of a driver;

FIG. 5 is a view for describing an example of changes in air blowing periods of air blasts in an awakening stimulation control portion;

FIG. 6 is a view for describing an example of changes in regions to which the cold air blasts are to be applied in the awakening stimulation control portion; and

FIG. 7 is a view for describing an example of changes in the regions to which the cold air blasts are to be applied in the awakening stimulation control portion.

### DETAILED DESCRIPTION

A conceivable technique changes a mode in which the stimulus for maintaining wakefulness is generated; such as alternate giving of the stimulus to the hands and the neck of the driver using the cold air blasts. However, in the conceivable technique, no measure is taken to reduce different feelings of discomfort experienced by individual drivers as a result of being exposed to the cold air blasts.

A wakefulness maintaining apparatus; a wakefulness maintaining method, and a non-transitory computer readable storage medium are provided each of which can further reduce different feelings of discomfort caused to individual target persons by a cold air blast for maintaining wakefulness.

According to an example embodiment, a wakefulness maintaining apparatus includes: an awakening stimulation controller that controls an awakening stimulation device to generate a cold air wind for maintaining a wakeful state of a target person; and a characteristic feature specification unit that specifies a characteristic feature of the target person. The awakening stimulation controller changes an air wind stimulation condition based on the characteristic feature of the target person specified by the characteristic feature specification unit. The air wind stimulation condition includes at least one of a region of the target person which the cold air wind generated from the awakening stimulation device hits, an air volume of the cold air wind, a temperature of the cold air wind, and an air blowing period of the cold air wind.

According to an example embodiment, a wakefulness maintaining method includes: specifying a characteristic feature of a target person; and changing at least one of a region of the target person which cold air wind hits, an air volume of the cold air wind, a temperature of the cold air wind, and an air blowing period of the cold air wind based on the specified characteristic feature of the target person. The cold air wind is generated by an awakening stimulation device for maintaining a wakeful state of the target person.

According to an example embodiment, a non-transitory computer readable storage medium includes instructions being executed by a computer. The instructions causes a computer to function as: a characteristic feature specification unit that specifies a characteristic feature of a target person; and an awakening stimulation controller that changes at least one of a region of the target person which cold air wind hits, an air volume of the cold air wind, a temperature of the cold air wind, and an air blowing period of the cold air wind based on the specified characteristic feature of the target person. The cold air wind is generated by an awakening stimulation device for maintaining a wakeful state of the target person.

According to the above apparatus, method and storage medium, based on the specified characteristic feature of the target person, at least any of the region of the target person to which the cold air blast generated from the awakening stimulation device is to be applied, the air volume of the cold air blast, the temperature thereof, and the air blowing period thereof is changed. Depending on the characteristic feature of the driver, the region of the driver to which the cold blast is to be applied and quantities such as the air volume of the cold air blast, the temperature thereof, and the air blowing period thereof may cause increased discomfort to the driver. Since at least any of the region of the target person to which the cold air blast is to be applied, the air volume of the cold air blast, the temperature thereof, and the air blowing period thereof is changed based on the specified characteristic feature of the target person, it is possible to change at least any of the region of the target person to which the cold air blast is to be applied, the air volume of the cold air blast, the temperature thereof, and the air blowing period thereof based on the characteristic feature of the target person, while reducing the probability of causing increased discomfort to the target person. This can further reduce different feelings of discomfort caused to individual target persons by the cold air blast for maintaining wakefulness.

### First Embodiment

<Schematic Configuration of Wakefulness Maintaining System 1>

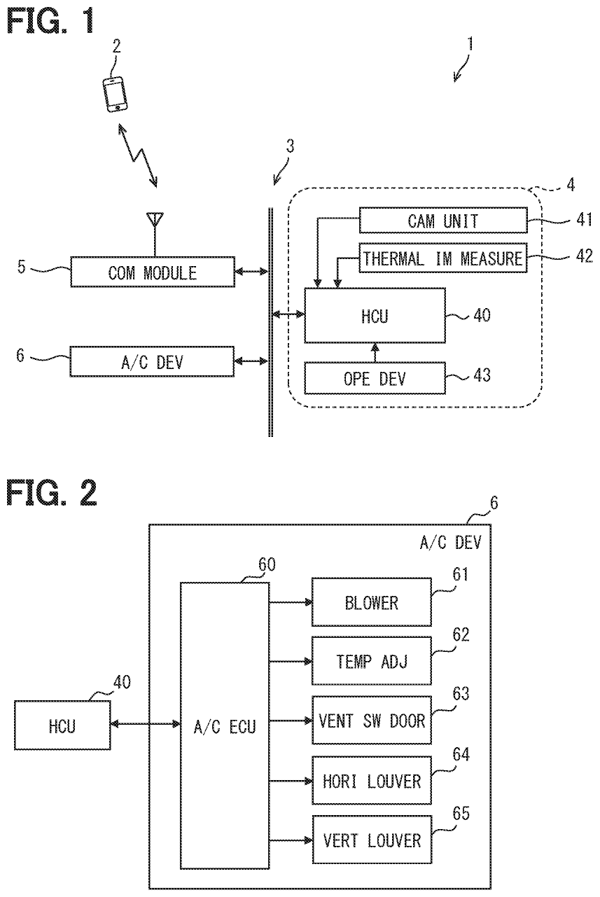

The following will describe a first embodiment using the drawings. A wakefulness maintaining system 1 shown in FIG. 1 includes a mobile terminal 2 and a vehicle-side system 3. It is assumed that the vehicle-side system 3 is to be used in an automobile (hereinafter referred to simply as the vehicle), while the mobile terminal 2 is to be carried by a driver of the vehicle.

The mobile terminal 2 may appropriately be a terminal which can be carried by a person and has a specific communication function and an input function. For example, a multifunctional mobile phone, a tablet terminal, a note PC, or the like can be used as the mobile terminal 2. By way of example, the following will describe a case where the mobile terminal 2 is a multifunctional mobile phone such as a smart phone and has, as the specific communication function, a communication function of performing communication based on a short-range wireless communication standard such as the Bluetooth Low Energy (Bluetooth is a registered trademark).

<Schematic Configuration of Vehicle-Side System 3>

As shown in FIG. 1, the vehicle-side system 3 includes a HMI (Human Machine Interface) system 4, a communication module 5, and an air conditioning device 6. It is assumed that the HMI system 4, the communication module 5, and the air conditioning device 6 are connected to, e.g., an in-vehicle LAN.

The HMI system 4 includes a HCU (Human Machine Interface Control Unit) 40, a camera unit 41, a thermal image measurement device 42, and an operation device 43. The HMI system 4 receives an input operation by the driver or monitors a state of the driver. The driver corresponds to a target person.

The camera unit 41 includes a near-infrared camera and a near-infrared light source. The near-infrared camera is a camera having a sensitivity to light in a near-infrared region. The near-infrared camera images an object to be imaged including a region illuminated with light from the near-infrared light source and outputs a captured image to the HCU 40. The near-infrared light source is an illuminating device which emits near-infrared light. Note that the near-infrared light source may also be another light source other than an LED, such as a light source having a filament or the like. The near-infrared camera captures an image (hereinafter referred to as the driver image) of a region of the driver occupying a driver seat including a facial region. In the first embodiment, the following description will be given using, as an example, a case where an image of the region of the driver including a face, hands, legs, and a chest is captured as the driver image. As for the legs, only portions of the legs such as, e.g., thigh portions may appropriately be included in the region to be imaged. The near-infrared camera is provided in, e.g., an upper surface of an instrument panel. Note that a position at which the near-infrared camera is provided is not limited to the upper surface of the instrument panel as long as the driver can be imaged at the position. For example, the near-infrared camera may also be configured to be provided on a steering column, in a roof, or in a rearview mirror.

The thermal image measurement device 42 measures a body surface temperature of the driver occupying the driver seat. By way of example, the thermal image measurement device 42 may appropriately be configured to output, as an image and to the HCU 40, a temperature distribution in a range corresponding to an upper body of the driver occupying the driver seat to be imaged using a thermographic camera. The thermal image measurement device 42 may also be configured such that the camera unit 41 is integrated with the thermal image measurement device 42 therein to allow the camera to be shared therebetween.

The operation device 43 is a group of switches to be operated by the driver to make various settings, Examples of the operation device 43 include a steering switch provided in a spoke portion of a steering wheel of a host vehicle and a touch switch integrated with a display.

The HCU 40 is configured to include, as a main component, a microcomputer including a processor, a memory, an I/O, and a bus connecting the processor, the memory, and the I/O, and executes a control program stored in the memory to thus perform various processes such as a process (hereinafter referred to as a wakefulness maintenance related process) related to maintenance of wakefulness. The execution of the control program by the processor corresponds to implementation of a wakefulness maintaining method corresponding to the control program. The memory mentioned herein is a non-transitory tangible storage medium which non-transitorily stores a computer-readable program and data. The non-transitory tangible storage medium is implemented by a semiconductor memory, a magnetic disk, or the like. The HCU 40 corresponds to a wakefulness maintaining apparatus. Note that details of a process performed in the HCU 40 will be described later.

The communication module 5 is connected to the mobile terminal 2 to be wirelessly communicative therewith using the short-range wireless communication standard mentioned above and performs communication. The communication module 5 outputs a signal received from the mobile terminal 2 to the HCU 40, and transmits information input thereto from the HCU 40 to the mobile terminal 2.

The air conditioning device 6 acquires air conditioning request information from the HCU 40 and performs adjustment (i.e., air conditioning) related to air blasts blown from a plurality of vents provided in the host vehicle. The air conditioning performed by the air conditioning device 6 includes adjustment of a volume of the blown air blast (hereinafter referred to as the air volume), adjustment of a temperature of the blown air blast, adjustment of an air blowing period of the blown air blast, adjustment of a direction of the blown air blast (hereinafter referred to as air direction), selection of the vents from which the air blasts are to be blown, and the like. The air conditioning device 6 corresponds to an awakening stimulation device. Details of the air conditioning device 6 will be described later.

It is assumed that the plurality of vents are provided at positions from which the air blasts can be blown toward the driver seat. In the first embodiment, by way of example, the description will be given continuously below on the assumption that, for the driver seat, a center face vent, a side face vent, and a foot vent are provided. For example, the center face vent is disposed in the vicinity of a middle portion of the instrument panel in a vehicle width direction so as to face the upper body of the driver occupying the driver seat. For example, the side face vent is disposed closer to a side window of the instrument panel so as to face the upper body of the driver occupying the driver seat. For example, the foot vent is disposed below a seat surface of the driver seat so as to face feet of the driver occupying the driver seat.

<Schematic Configuration of Air Conditioning Device 6>

As shown in FIG. 2, the air conditioning device 6 includes an air conditioning ECU 60, a blower 61, a temperature adjustment portion 62, a vent switching door 63, a horizontal louver 64, and a vertical louver 65.

The blower 61 adjusts the air volume of the air blast blown from each of the vents under the control of the air conditioning ECU 60. The temperature adjustment portion 62 includes a heat exchanger, an air mix damper, or the like and adjusts the temperature of the air blast blown from each of the vents under the control of the air conditioning ECU 60.

The vent switching door 63 switches the vent from which the air blast is to be blown under the control of the air conditioning ECU 60. By way of example, the vent switching door 63 switches the vent from which the air blast is to be blown to any of the center face vent, the side face vent, and the foot vent.

The horizontal louver 64 is provided upstream of the center face vent and the side face vent along a flow path of each of the air blasts and in the vicinity of the center face vent and the side face vent. The horizontal louver 64 includes a plurality of elongated plates formed so as to extend in the vehicle width direction of the vehicle. The plurality of elongated plates are arranged to be spaced apart from each other in a height direction of the vehicle. Each of the elongated plates of the horizontal louver 64 is disposed to be rotatably driven around a shaft thereof extending in the vehicle width direction by a motor for driving the shafts.

The vertical louver 65 is also provided upstream of the center face vent and the side face vent along the flow path of each of the air blasts and in the vicinity of the center face vent and the side face vent. The vertical louver 65 includes a plurality of elongated plates formed so as to extend in the height direction of the vehicle. The plurality of elongated plates are arranged to be spaced apart from each other in the width direction of the vehicle. Each of the elongated plates of the vertical louver 65 is disposed to be rotatably driven around a shaft thereof extending in the height direction of the host vehicle by the motor for driving the shafts.

Under the control of the air conditioning ECU 60, the horizontal louver 64 for the center face vent and the side face vent is rotated to allow a vertical direction of the air blast blown from each of the center face vent and the side face vent to be adjusted. Also, under the control of the air conditioning ECU 60, the vertical louver 65 for the center face vent and the side face vent is rotated to allow a lateral direction of the air blast blown from each of the center face vent and the side face vent to be adjusted.

The air conditioning device 6 switches the vent from which the air blast is to be blown using the vent switching door 63 and also adjusts the air directions of the air blast blown from each of the center face vent and the side face vent using the horizontal louver 64 and the vertical louver 65 to thus adjust the region of the driver occupying the driver seat to which the air blast is to be applied.

For example, it is appropriate to adjust the region to which the air blast is to be applied to the face by causing the air blast to be blown from each of the center face vent and the side face vent, bringing the lateral air direction of the air blast blown from each of the center face vent and the side face vent closer to a center of the driver seat, and bringing the vertical air direction of the air blast blown therefrom closer to a head rest of the driver seat. It is also appropriate to adjust the region to which the air blast is to be applied to the chest by causing the air blast to be blown from each of the center face vent and the side face vent, bringing the lateral air direction of the air blast blown from each of the center face vent and the side face vent closer to the center of the driver seat, and bringing the vertical air direction of the air blast blown therefrom closer to the center of the driver seat. It is also appropriate to adjust the region to which the air blast is to be applied to the hands by causing the air blast to be blown from each of the center face vent and the side face vent, bringing the lateral air direction of the air blast blown from the center face vent closer to a left arm rest of the driver seat, bringing the lateral air direction of the air blast blown from the side face vent closer to a right arm rest of the driver seat, and bringing the vertical air direction of the air blast blown therefrom closer to the center of the driver seat. Besides, it is appropriate to adjust the region to which the air blast is to be applied to the legs by causing the air blast to be blown from the foot vent.

In addition, under the control of the air conditioning ECU 60, the air conditioning device 6 also changes the timing of adjustment of the region to which the air blast is to be applied described above to thus adjust the air blowing period of the air blast to be applied to each of the regions.

The air conditioning ECU 60 is configured to include, as a main component, a microcomputer including a processor, a memory, an I/O, and a bus connecting the processor, the memory, and the I/O. The air conditioning ECU 60 executes a control program stored in the memory to thus perform various processes related to air conditioning. The air conditioning ECU 60 acquires air conditioning request information output from the HCU 40 and based on the acquired air conditioning request information, adjusts the air volume of the air blast, the temperature thereof, the air blowing period thereof, and the region to which the air blast is to be applied.

<Schematic Configuration of HCU 40>

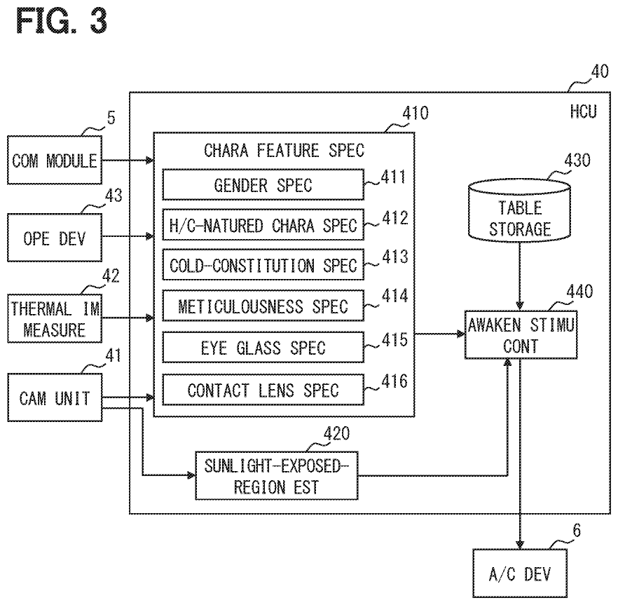

Subsequently, using FIG. 3, a description will be given of a schematic configuration of the HCU 40. The HCU 40 includes a characteristic feature specification portion 410, a sunlight-exposed-region estimation portion 420, a table storage portion 430, and an awakening stimulation control portion 440. The characteristic feature specification portion 410 includes a gender specification portion 411, a hot/cold-natured characteristic specification portion 412, a cold constitution (i.e., cold intolerance) specification portion 413, a meticulousness specification portion 414, an eyeglass specification portion 415, and a contact lens specification portion 416. Note that some or all of the functions performed by the HCU 40 may also be configured using hardware such as one or a plurality of ICs. Also, some or all of functional blocks included in the HCU 40 may also be implemented by a combination of execution of software by a processor and a hardware member.

The characteristic feature specification portion 410 specifies a characteristic feature of the driver. Examples of the characteristic feature of the driver to be specified by the characteristic feature specification portion 410 include gender, being hot/cold-natured, having cold intolerance, being meticulous, wearing eyeglasses, and wearing contact lenses. The gender specification portion 411 specifies the gender as the characteristic feature of the driver. The hot/cold-natured characteristic specification portion 412 specifies being hot/cold-natured as the characteristic feature of the driver. The cold constitution specification portion 413 specifies having cold intolerance as the characteristic feature of the driver. The meticulousness specification portion 414 specifies being meticulous as the characteristic feature of the driver. The eyeglass specification portion 415 specifies wearing eyeglasses as the characteristic feature of the driver. The contact lens specification portion 416 specifies wearing contact lenses as the characteristic feature of the driver.

The gender specification portion 411 may appropriately be configured to perform an image recognition process, such as pattern matching, on the driver image output from the camera unit 41 and specify whether the gender of the driver is male or female. Alternatively, the gender specification portion 411 may also be configured to receive an input indicative the gender of the driver, which is either male or female, via the mobile terminal 2 or the operation device 43, acquire the input result via the HCU 40, and specify whether the gender of the driver is male or female based on the input result. The input result from the mobile terminal 2 may appropriately be acquired by the HCU 40 via the communication module 5.

The hot/cold-natured characteristic specification portion 412 may appropriately be configured to receive an input indicative of whether the driver is hot-natured, cold-natured, or neither hot-natured nor cold-natured via the mobile terminal 2 or the operation device 43, acquire the input result via the HCU 40, and specify whether the driver is hot-natured, cold-natured, or neither hot-natured nor cold-natured based on the input result. In the case of a configuration which allows an air conditioning temperature of the air conditioning device 6 to be remotely set via the mobile terminal 2, the hot/cold-natured characteristic specification portion 412 may also be configured to specify whether the driver is hot-natured or cold-natured based on whether the air conditioning temperature set via the mobile terminal 2 is lower or higher than a reference temperature by a prescribed value or more, Otherwise, the hot/cold-natured characteristic specification portion 412 may also be configured to specify whether the driver is hot-natured or cold-natured using another method such as specifying that the driver is hot-natured when a height and a weight each input to the mobile terminal 2 is equal to or more than prescribed values. The hot/cold-natured characteristic specification portion 412 may also be configured such that the mobile terminal 2 estimates whether the driver is hot-natured or cold-natured, and the hot/cold-natured characteristic specification portion 412 specifies whether the driver is hot-natured or cold-natured based on a result of the estimation.

The cold constitution specification portion 413 may appropriately be configured to specify, based on a result of measuring the body surface temperature of the driver which is output from the thermal image measurement device 42, whether or not the driver has cold intolerance. By way of example, the cold constitution specification portion 413 may appropriately specify whether or not the driver has cold intolerance based on whether or not the body surface temperature of a distal end portion of the driver, such as a hand, is equal to or less than a threshold for determining whether or not the driver has cold intolerance. Alternatively, the cold constitution specification portion 413 may also be configured appropriately to receive an input indicative of whether or not the driver has cold intolerance via the mobile terminal 2 or the operation device 43, acquire the input result via the HCU 40, and specify whether or not the driver has cold intolerance based on the input result.

The meticulousness specification portion 414 may appropriately be configured to receive an input indicative of whether or not the driver is meticulous via the mobile terminal 2 or the operation device 43, acquire the input result via the HCU 40, and specify whether or not the driver is meticulous based on the input result. In the case of a configuration which allows the mobile terminal 2 to use a social network, the meticulousness specification portion 414 may also be configured to specify whether or not the driver is meticulous based on the content of a message from the driver as the user of the mobile terminal 2 in the social network. By way of example, the meticulousness specification portion 414 may appropriately analyze the frequency of the use of phrases characteristic to a meticulous person using text mining or the like and thus specify whether or not the driver is meticulous. The meticulousness specification portion 414 may also be configured such that the mobile terminal 2 estimates whether or not the driver is meticulous, and the meticulousness specification portion 414 specifies whether or not the driver is meticulous based on a result of the estimation.

The eyeglass specification portion 415 may appropriately be configured to perform an image recognition process, such as pattern matching, on the driver image output from the camera unit 41 and thus specify whether or not the driver is wearing eyeglasses. Alternatively, the eyeglass specification portion 415 may also be configured to receive an input indicative of whether or not the driver is waiting eyeglasses via the mobile terminal 2 or the operation device 43, acquire the input result via the HCU 40, and specify whether or not the driver is wearing eyeglasses based on the input result.

The contact lens specification portion 416 may appropriately be configured to extract eye regions from the driver image output from the camera unit 41 and specify, based on a reflectance of light from the eye regions, whether or not the driver is wearing contact lenses. The contact lens specification portion 416 may also receive an input indicative of whether or not the driver is wearing contact lenses via the mobile terminal 2 or the operation device 43, acquire the input result via the HCU 40, and specify whether or not the driver is wearing contact lenses based on the input result.

To reduce the labor of specifying the characteristic feature of the driver, except for the characteristic feature of wearing eyeglasses or wearing contact lenses, every time the driver gets into the host vehicle, it may be appropriate to, e.g., store the characteristic features of the different drivers specified by the characteristic feature specification portion 410 in association with the IDs of electronic keys used for authentication in the nonvolatile memory of the HCU 40 and thus individually store the characteristic features of the different drivers. The HCU 40 may appropriately be configured to read, every time a driver gets into the vehicle, the characteristic feature corresponding to the ID of the electronic key used for authentication, and use the read characteristic feature as the characteristic feature of the driver.

In the configuration shown herein, to allow the characteristic feature specification portion 410 to specify the characteristic feature of the driver, the communication module 5, the camera unit 41, the thermal image measurement device 42, and the operation device 43 are used. However, a configuration to be used is not limited thereto. Depending on a method of specifying the characteristic feature of the driver to be used, the use of an unrequired configuration may appropriately be omitted.

The sunlight-exposed-region estimation portion 420 estimates the region of the driver exposed to sunlight. By way of example, the sunlight-exposed-region estimation portion 420 performs an image recognition process, such as pattern matching, on the driver image output from the camera unit 41 and thus distinctively extracts each of the regions of the driver such as, e.g., the hands, the legs, the face, and the chest. Then, the sunlight-exposed-region estimation portion 420 may appropriately estimate the region exposed to sunlight based on whether or not a brightness of each of the regions is equal to or more than a threshold for determining whether or not the region is exposed to sunlight.

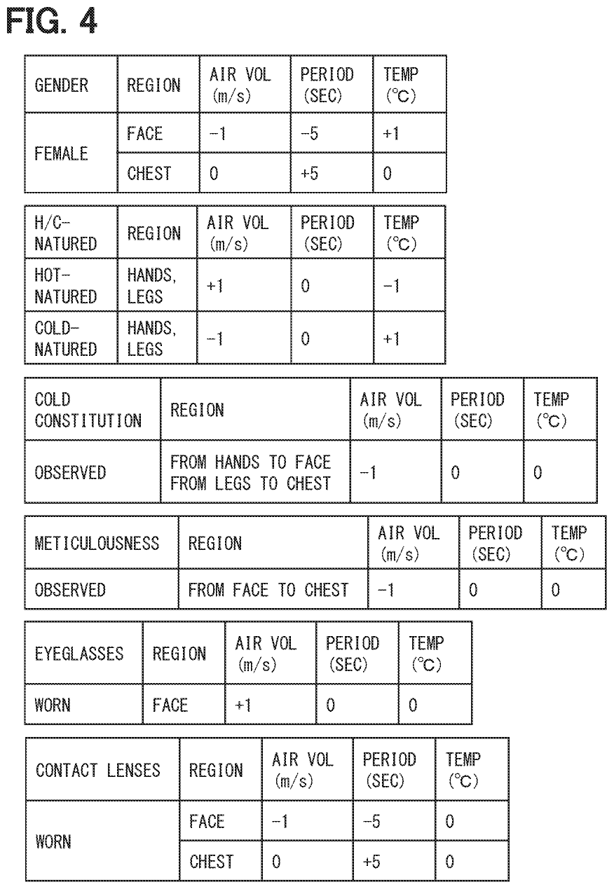

The table storage portion 430 stores respective tables in each of which one of the characteristic features of the driver is associated with a change of the region of the driver to which the cold air blast from the air conditioning device 6 is to be applied and with set values indicative of respective amounts of change in quantitative conditions for the individual regions to which the cold air blast is to be applied, such as the air volume of the cold air blast, the temperature thereof, and the air blowing period thereof. The table storage portion 430 may appropriately be configured to use a nonvolatile memory. Using FIG. 4, a description will be given herein of an example of the tables stored in the table storage portion 430. It is assumed that, in the following description, a unit of the air volume is m/s, a unit of the temperature is ° C., and a unit of the air blowing period is a second.

As shown in FIG. 4, the characteristic feature of female gender is associated with the set values indicative of a 1 m/s air volume reduction, a 1° C. temperature increase, and a 5 second air blowing period reduction for the cold air blast to be applied to the face, while being associated with the set value indicative of a 5 second air blowing period increase for the cold air blast to be applied to the chest. It is assumed that, for the characteristic feature of male gender for which a table about changes is not shown, the region to which the cold air blast is to be applied is not changed from a default region, and the quantitative conditions such as the air volume, the temperature, and the air blowing period are also not changed from default reference values. Note that the region to which the cold air blast is to be applied is successively changed in a default order of the face, the hands, the chest, and the legs in one cycle. In addition, the reference values are the air volume of the cold air blast, the temperature thereof, and the air blowing period thereof each for maintaining the wakeful state of the driver, which are uniformly determined in advance irrespective of the characteristic features of the drivers. The reference values may appropriately be, e.g., respective representative values, such as an average value or a mode value, of the air volume of the cold air blast, the temperature thereof, and the air blowing period thereof which were effective in maintaining the wakeful states of a plurality of test subjects. In the first embodiment, by way of example, a description will be given below of a case where the reference values of the air volume, the temperature, and the air blowing period are 2 m/s, 15° C., and 15 seconds.

As shown in FIG. 4, the characteristic feature of being hot-natured included in the characteristic feature of being hot/cold-natured is associated with the set values indicative of a 1 m/s air volume increase and a 1° C. temperature reduction for each of the cold air blasts applied to the hands and the legs. The characteristic feature of being cold-natured included in the characteristic feature of being hot/cold-natured is associated with the set values indicative of a 1 m/s air volume reduction and a 1° C. temperature increase for each of the cold air blasts applied to the hands and the legs. When the driver is neither hot-natured nor cold-natured, the region to which the cold air blast is to be applied is not changed from the default region, and the quantitative conditions such as the air volume, the temperature, and the air blowing period are also not changed from the default reference values.

As shown in FIG. 4, the characteristic feature of having cold intolerance is associated with a change of the region to which the cold air blast is to be applied from the hands to the face, a change of the region to which the cold air blast is to be applied from the legs to the chest, and the set value indicative of a 1 m/s air volume reduction for the cold air blast. When the driver does not have cold intolerance, the region to which the cold air blast is to be applied is not changed from the default region, and the quantitative conditions such as the air volume, the temperature, and the air blowing period are also not changed from the default reference values.

As shown in FIG. 4, the characteristic feature of being meticulous is associated with a change of the region to which the cold air blast is to be applied from the face to the chest and the set value indicative of a 1 m/s air volume reduction for the cold air blast. When the characteristic feature of the driver is not being meticulous, the region to which the cold air blast is to be applied is not changed from the default region, and the quantitative conditions such as the air volume, the temperature, and the air blowing period are also not changed from the default reference values.

As shown in FIG. 4, the characteristic feature of wearing eyeglasses is associated with the set value indicative of a 1 m/s air volume increase for the cold air blast to be applied to the face. The characteristic feature of wearing contact lenses is associated with the set values indicative of a 1 m/s air volume reduction and a 5 second air blowing period increase for the cold air blast to be applied to the face, while being also associated with the set value indicative of a 5 second air blowing period increase for the cold air blast to be applied to the chest. When the characteristic feature of the driver is neither wearing eyeglasses nor wearing contact lenses, the region to which the cold air blast is to be applied is not changed from the default region, and the quantitative conditions such as the air volume, the temperature, and the air blowing period are also not changed from the default reference values.

The awakening stimulation control portion 440 causes the air conditioning device 6 to generate a cold air blast for maintaining the wakeful state of the driver. The awakening stimulation control portion 440 outputs air conditioning request information toward the air conditioning ECU 60 of the air conditioning device 6 to control the operation thereof. A process of causing the cold air blast for maintaining the wakeful state of the driver to be generated, which is performed by the awakening stimulation control portion 440, is hereinafter referred to as an awakening stimulation process.

For example, the awakening stimulation control portion 440 may appropriately be configured to start the awakening simulation process when an ignition power source of the host vehicle is ON and a power source of the HCU 40 is also ON. Alternatively, the awakening stimulation control portion 440 may also be configured to start the awakening simulation process when a degree of wakefulness of the driver sensed based on the driver image output from the camera unit 41 is equal to or less than a prescribed level. Still alternatively, the awakening stimulation control portion 440 may also be configured to start the awakening simulation process when a continuous drive time or a travel distance from the time when the host vehicle started driving is equal to or more than a given value. On the other hand, the awakening stimulation control portion 440 may appropriately end the awakening stimulation process when the ignition power source of the host vehicle is OFF and the power source of the HCU 40 is also OFF or end the awakening stimulation process when the degree of wakefulness of the driver mentioned above is equal to or more than a predetermined level higher than the prescribed level mentioned above.

Preferably, in the awakening stimulation process, the awakening stimulation control portion 440 successively changes the region of the driver to which the cold air blast is to be applied from one region to another in rotation. This is intended to reduce the probability that the driver becomes accustomed to the cold air blast by successively changing the region to which the cold air blast is to be applied from one region to another and enhance an effect of maintaining wakefulness. In the first embodiment, by way of example, a description will be given of a case where, in a default awakening stimulation process, a cycle is repeated in which the region to which the cold air blast is to be applied is successively changed in order of the face, the hands, the chest, and the legs in rotation.

In the awakening stimulation process, the awakening stimulation control portion 440 changes, based on the characteristic feature of the driver specified by the characteristic feature specification portion 410, an air blast stimulation condition which is at least any of the region of the driver to which the cold air blast generated by the air conditioning device 6 is to be applied, the air volume of the cold air blast, the temperature thereof, and the air blowing period thereof. The awakening stimulation control portion 440 may appropriately be configured to change the quantitative conditions such as the air volume of the cold air blast, the temperature thereof, and the air blowing period thereof from the respective reference values. More specifically, the awakening stimulation control portion 440 changes the quantitative conditions to values obtained by adding or subtracting, to or from the respective reference values, such set values as shown in FIG. 4 in accordance with the characteristic feature of the driver specified by the characteristic feature specification portion 410. In the first embodiment, by way of example, a description will be given below of a case where the awakening stimulation control portion 440 causes the air blast stimulation condition in accordance with the characteristic feature of the driver to be changed based on the tables in FIG. 4.

When the characteristic feature specification portion 410 specifies the plurality of characteristic features, it may be appropriate to change the quantitative conditions to values obtained by adding or subtracting, to or from the respective reference values, such set values as shown in FIG. 4 in accordance with the plurality of characteristic features. When amounts in which the quantitative conditions are to be changed are to be adjusted by allowing one graph to represent the plurality of characteristic features, parameters become complicated. However, with a configuration which changes the quantitative conditions to values obtained by adding or subtracting, to or from the respective reference values, the set values in accordance with the plurality of characteristic features, it is sufficient only to add or subtract the set values. This further simplifies adjustment of the amounts in which the quantitative conditions are to be changed.

Each of the quantitative conditions to be changed by the awakening stimulation control portion 440 has a maximum value and a minimum value which are set in advance within a range narrower than the range that can be set by the air conditioning device 6. When each of the quantitative conditions is to be changed, the awakening stimulation control portion 440 preferably changes the quantitative conditions within the range not more than the maximum value and not less than the minimum value. The awakening stimulation control portion 440 may appropriately be configured as follows. For example, when a value obtained by adding or subtracting, to or from the reference value, each of the set values in accordance with the plurality of characteristic features is more than the maximum value, the awakening stimulation control portion 440 changes each of the quantitative conditions to the maximum value. On the other hand, when the value obtained by adding or subtracting, to or from the reference value, each of the set values in accordance with the plurality of characteristic features is less than the minimum value, the awakening stimulation control portion 440 changes each of the quantitative conditions to the minimum value.

This is because, when the temperature of the cold air blast becomes excessively high, the effect of maintaining the wakefulness of the driver decreases while, when the temperature of the cold air blast becomes excessively low or the air volume of the cold air blast becomes excessively large, discomfort felt by the driver increases irrespective of the characteristic features of the drivers. Accordingly, as the maximum value and the minimum value, a value falling within a range estimated to be effective in maintaining the wakeful state of the driver and a value less than a range estimated to increase the discomfort felt by the driver irrespective of the characteristic features of the drivers may be set appropriately. By way of example, the quantitative conditions to be changed by the awakening stimulation control portion 440 may appropriately be such that the air volume of the cold air blast ranges from 3 m/s to 1 m/s, the temperature thereof ranges from 20° C. to 10° C., and the air blowing period thereof ranges from 30 seconds to 10 seconds.

Alternatively, the awakening stimulation control portion 440 may also be configured to select, from among the plurality of characteristic features of the driver specified by the characteristic feature specification portion 410, a predetermined number of the characteristic features in order of priority and change the quantitative conditions to values obtained by adding or subtracting, to or from respective reference values, set values in accordance with the predetermined number of selected characteristic features. For example, the HCU 40 may appropriately be configured such that tables in which the individual characteristic features are associated with the respective priorities are stored in advance in the nonvolatile memory of the HCU 40 to allow the awakening stimulation control portion 440 to refer to the tables based on the characteristic features and specify the priority of each of the characteristic features. The predetermined number mentioned herein is an arbitrarily settable value.

This allows the set values for the characteristic feature having the higher priority to be preferentially reflected on the adjustment of the cold air blast. To allow the discomfort caused by the cold air blast in the driver to be further reduced, the characteristic feature with respect to which the cold air blast is more likely to affect a feeling of discomfort preferably has a higher priority. With respect to the characteristic feature of being hot/cold-natured, the cold air blast is particularly likely to affect a feeling of discomfort, and accordingly the priority of the characteristic feature of being hot/cold-natured is preferably set highest.

Alternatively, the awakening stimulation control portion 440 may also be configured to determine the respective priorities of the individual characteristic features according to a season. The awakening stimulation control portion 440 may appropriately change the respective priorities of the individual characteristic features according to the current season, select, from among the plurality of characteristic features of the driver specified by the characteristic feature specification portion 410, the predetermined number of the characteristic features in order of priority, and change the quantitative conditions to values obtained by adding or subtracting, to or from the respective reference values, the set values in accordance with the predetermined number of selected characteristic features. The current season may appropriately be specified by the awakening stimulation control portion 440 based on a time and date measured by a measuring means.

It may be considered that the characteristic features with respect to which the cold air blast is more likely to affect a feeling of discomfort change according to the season. Therefore, by changing the respective priorities of the individual characteristic features according to the current season, it is possible to more accurately reduce the discomfort felt by the driver due to the cold air blast. When the respective priorities of the individual characteristic features are determined based on the season, it may be appropriate to, e.g., increase the priorities of the characteristic features of having cold intolerance and being hot/cold-natured in winter and increase the priorities of the characteristic features of gender and being meticulous in summer.

When the gender specification portion 411 specifies the characteristic feature of the driver as female, the awakening stimulation control portion 440 increases the air volume of the cold air blast and reduces the temperature thereof as compared to those when the gender specification portion 411 specifies the characteristic feature of the driver as male. In addition, the awakening stimulation control portion 440 reduces the air blowing period of the cold air blast applied to the face, while correspondingly increasing the air blowing period of the cold air blast applied to the chest. When the characteristic feature of the driver is male, the quantitative conditions are not changed from the reference values while, when the characteristic feature of the driver is female, the air volume of the cold air blast applied to the face is reduced by 1 m/s from the reference value, the temperature thereof is increased by 1° C., the air blowing period thereof is reduced by 5 seconds. On the other hand, the air blowing period of the cold air blast applied to the chest is increased by 5 seconds.

In the configuration described above, the air volume and the air blowing period of the cold air blast applied to the face of a female are reduced, while the temperature of the cold air blast applied to the face of the female is increased. Thus, it is possible to reduce the discomfort felt by the female who tends to feel uncomfortable with the cold air blast applied to her face. Since a female tends to be highly sensitive to the cold air blast applied to her face, even when the air volume of the cold air blast applied to the face is reduced and the temperature thereof is increased, the effect of maintaining wakefulness is likely to be maintained. In addition, while the air blowing period of the cold air blast applied to the face is reduced, the air blowing period of the cold air blast applied to the chest is correspondingly increased. In this point also, the effect of maintaining wakefulness is likely to be maintained. Moreover, the region to which the cold air blast is to be applied is changed not to distal ends such as the hands or legs, but to the chest. Consequently, it is also possible to prevent a female who tends to have cold intolerance in distal ends from being excessively cooled, while changing the region to which the cold air blast is to be applied from the face and continuously providing the effect of maintaining wakefulness.

In the case of using a configuration which selects, from among the plurality of characteristic features of the driver, a predetermined number of the characteristic features in order of priority, it is appropriate to take the following procedure. When the gender specification portion 411 specifies the characteristic feature of the driver as female and the awakening stimulation control portion 440 selects the characteristic feature of female gender based on the priority, the awakening stimulation control portion 440 may appropriately increase the air volume of the cold air blast, reduce the temperature thereof, and reduce the air blowing period of the air blast applied to the face, while increasing the air blowing period of the cold air blast applied to the chest, as compared to those when the gender specification portion 411 specifies the characteristic feature of the driver as male.

When the hot/cold-natured characteristic specification portion 412 specifies the characteristic feature of the driver as being hot-natured, the awakening stimulation control portion 440 reduces the temperature of the cold air blast as compared to that when the hot/cold-natured characteristic specification portion 412 specifies the characteristic feature of the driver as neither being hot-natured nor being cold-natured. In addition, the awakening stimulation control portion 440 increases the air volume of the cold air blast, while reducing the temperature thereof. For example, since the distal ends such as the hands or legs tend to be highly sensitive to the temperature, to allow the driver to easily recognize that the temperature of the cold air blast is improved, it is preferable to reduce the temperature of the cold air blast applied to each of the hands and the legs and increase the air volume thereof. When the characteristic feature of the driver is neither being hot-natured nor being cold-natured, the quantitative conditions are not changed from the reference values. On the other hand, when the driver is hot-natured, the air volume of the cold air blast applied to each of the hands and the legs is increased by 1 m/s from the reference value and the temperature thereof is reduced by 1° C. from the reference value.

With the configuration described heretofore, the temperature of the cold air blast applied to the hot-natured driver is reduced, while the air volume thereof is increased. This can change the cold air blast such that the driver is more likely to feel comfortable and thus reduce discomfort. In addition, the temperature of the cold air blast applied to each of the hands and the legs is reduced, while the air volume thereof is increased. This allows the driver to easily recognize the improvements and allows for a further reduction in discomfort.

In the case of using a configuration which selects, from among the plurality of characteristic features of the driver, a predetermined number of the characteristic features in order of priority, it is appropriate to follow the following procedure. When the hot/cold-natured characteristic specification portion 412 specifies the characteristic feature of the driver as being hot-natured and the awakening stimulation control portion 440 selects the characteristic feature of being hot-natured based on the priority, the awakening stimulation control portion 440 may appropriately reduce the temperature of the cold air blast, while increasing the air volume thereof.

When the hot/cold-natured characteristic specification portion 412 specifies the characteristic feature of the driver as being cold-natured, the awakening stimulation control portion 440 increases the temperature of the cold air blast as compared to that when the hot/cold-natured characteristic specification portion 412 specifies the characteristic feature of the driver as neither being hot-natured nor being cold-natured. In addition, the awakening stimulation control portion 440 reduces the air volume of the cold air blast, while increasing the temperature thereof. For example, to allow the driver to easily recognize an improvement in the temperature of the cold air blast, it is preferable to increase the temperature of the cold air blast applied to each of the hands and the legs and reduce the air volume thereof. When the characteristic feature of the driver is neither being hot-natured nor being cold-natured, the quantitative conditions are not changed from the reference values. On the other hand, when the driver is cold-natured, the air volume of the cold air blast applied to each of the hands and the legs is reduced by 1 m/s from the reference value and the temperature thereof is increased by 1° C. from the reference value.

With the configuration described heretofore, the temperature of the cold air blast applied to the cold-natured driver is increased, while the air volume thereof is reduced. This can change the cold air blast such that the driver is more likely to feel comfortable and thus reduce discomfort. In addition, the temperature of the cold air blast applied to each of the hands and legs is increased, while the air volume thereof is reduced. This allows the driver to easily recognize the improvements and allows for a further reduction in discomfort.

In the case of using a configuration which selects, from among the plurality of characteristic features of the driver, a predetermined number of the characteristic features in order of priority, it is appropriate to follow the following procedure. When the hot/cold-natured characteristic specification portion 412 specifies the characteristic feature of the driver as being cold-natured and the awakening stimulation control portion 440 selects the characteristic feature of being cold-natured based on the priority, the awakening stimulation control portion 440 may appropriately increase the temperature of the cold air blast, while reducing the air volume thereof.

When the cold constitution specification portion 413 does not specify the characteristic feature of the driver as having cold intolerance, the awakening stimulation control portion 440 assumes that the hands, the legs, and the other regions of the driver are regions to which the cold air blasts are to be applied. On the other hand, when the characteristic feature specified by the cold constitution specification portion 413 is having cold intolerance, the awakening stimulation control portion 440 assumes that, among the hands, the legs, and the other regions of the driver, the hands and the legs are regions to which the cold air blasts are not to be applied. In addition, while assuming that the hands and the legs are the regions to which the cold air blasts are not to be applied, the awakening stimulation control portion 440 causes the cold air blasts to be applied to the regions other than the hands and the legs and reduces the air volume thereof. When the characteristic feature of the driver is not having cold intolerance, the awakening stimulation control portion 440 does not change the quantitative conditions from the reference values. On the other hand, when the characteristic feature of the driver is having cold intolerance, the awakening stimulation control portion 440 changes the region to which the cold air blast is to be applied from the hands to the face and from the legs to the chest, and reduces the air volume of the cold air blast applied to each of the regions by 1 m/s from the reference value. Note that the awakening stimulation control portion 440 is not limited to the configuration which reduces the air volume of the cold air blast applied to each of the regions. The awakening stimulation control portion 440 may also be configured to reduce the air volume of the cold air blast applied to each of the regions which is exposed to the cold air blast in place of the hands and the legs.

With the configuration described heretofore, it is possible stop the application of the cold air blasts to the distal ends, such as the hands and the legs, of the driver suffering from cold intolerance which tend to have cold intolerance, render the body of the driver less likely to be chilled, and reduce discomfort. In addition, since the air volume of the cold air blast applied to each of the regions is reduced, it is possible to accordingly render the body of the driver less likely to be chilled and reduce discomfort. Besides, while the application of the cold air blast to each of the hands and the legs is stopped, the cold air blast is correspondingly applied to the face and the chest so that the effect of maintaining wakefulness is likely to be maintained.

In the case of using a configuration which selects, from among the plurality of characteristic features of the driver, a predetermined number of the characteristic features in order of priority, it is appropriate to take the following procedure. When the cold constitution specification portion 413 specifies the characteristic feature of the driver as having cold intolerance and the awakening stimulation control portion 440 selects the characteristic feature of having cold intolerance based on the priority, the awakening stimulation control portion 440 may appropriately assume that, among the hands, the legs, and the other regions of the driver, the hands and the legs are the regions to which the cold air blasts are not to be applied or reduce the volumes of the air blasts applied thereto.

When the characteristic feature of the driver specified by the meticulousness specification portion 414 is being meticulous, the awakening stimulation control portion 440 reduces the air volume of the cold air blast as compared to that when the meticulousness specification portion 414 does not specify the characteristic feature of the driver as being meticulous. In addition, the awakening stimulation control portion 440 changes the region to which the cold air blast is to be applied from the face to the chest. When the characteristic feature of the driver is not being meticulous, the awakening stimulation control portion 440 does not change the quantitative conditions from the reference values. On the other hand, when the characteristic feature of the driver is being meticulous, the awakening stimulation control portion 440 changes the region to which the cold air blast is to be applied from the face to the chest and also reduces the air volume of the cold air blast applied to each of the regions by 1 m/s from the reference value. Note that the awakening stimulation control portion 440 is not limited to the configuration which reduces the air volume of the cold air blast applied to each of the regions. The awakening stimulation control portion 440 may also be configured to reduce the air volume of the cold air blast applied to the chest which is exposed to the cold air blast instead of the face.

With the configuration described heretofore, the air volume of the cold air blast applied to each of the regions of the meticulous driver who is considered to be hypersensitive to the air blast is reduced. Accordingly, it is possible to reduce discomfort. In addition, since the application of the cold air blast to the face considered to be particularly hypersensitive to the air blast is stopped and the region to which the cold air blast is to be applied is changed to the chest, it is possible to accordingly reduce discomfort. Moreover, since the air volume of the cold air blast applied to each of the regions is also reduced, it is possible to accordingly render the body of the driver less likely to be chilled and reduce discomfort. Besides, while the application of the cold air blast to the face is stopped, the cold air blast is correspondingly applied to the chest, so that the effect of maintaining wakefulness is likely to be maintained. In addition, since the meticulous driver is considered to be hypersensitive to the air blast, even when the air volume of the cold air blast applied to each of the regions is reduced, the effect of maintaining wakefulness is likely to be maintained.

In the case of using a configuration which selects, from among the plurality of characteristic features of the driver, a predetermined number of the characteristic features in order of priority, it is appropriate to take the following procedure. When the meticulousness specification portion 414 specifies the characteristic feature of the driver as being meticulous and the awakening stimulation control portion 440 selects the characteristic feature of being meticulous based on the priority, the awakening stimulation control portion 440 may appropriately reduce the air volume or change the region to which the cold air blast is to be applied from the face to the region other than the face.

When the characteristic feature of the driver specified by the eyeglass specification portion 415 is wearing eyeglasses, the awakening stimulation control portion 440 increases the air volume of the cold air blast applied to the face as compared to that when the eyeglass specification portion 415 does not specify the characteristic feature of the driver as wearing eyeglasses. When the characteristic feature of the driver is not wearing eyeglasses, the awakening stimulation control portion 440 does not change the air volume of the cold air blast applied to the face from the reference value. On the other hand, when the characteristic feature of the driver is wearing eyeglasses, the awakening stimulation control portion 440 increases the air volume of the cold air blast applied to the face by 1 m/s from the reference value.

With the configuration described heretofore, even when the effect of maintaining wakefulness is enhanced by increasing the air volume of the cold air blast applied to the face of the driver wearing eyeglasses, having the eyes less likely to be exposed to the cold air blast, and less likely to feel uncomfortable with the cold air blast, it is hard to reduce the discomfort felt by the driver. In the case of using a configuration which selects, from among the plurality of characteristic features of the driver, a predetermined number of the characteristic features in order of priority, it is appropriate to take the following procedure. When the eyeglass specification portion 415 specifies the characteristic feature of the driver as wearing eyeglasses and the awakening stimulation control portion 440 selects the characteristic feature of wearing eyeglasses based on the priority, the awakening stimulation control portion 440 may appropriately increase the air volume of the air blast applied to the face.

When the characteristic feature of the driver specified by the contact lens specification portion 416 is wearing contact lenses, the awakening stimulation control portion 440 reduces the air volume applied to the face as compared to that when the contact lens specification portion 416 does not specify the characteristic feature of the driver as wearing contact lenses. The awakening stimulation control portion 440 also reduces the air blowing period of the cold air blast applied to the face, while correspondingly increasing the air blowing period of the cold air blast applied to the chest. When the driver is not wearing contact lenses, the awakening stimulation control portion 440 does not change the quantitative conditions from the reference values. On the other hand, when the characteristic feature of the driver is wearing contact lenses, the awakening stimulation control portion 440 reduces the air volume of the cold air blast applied to the face by 1 m/s from the reference value, and also reduces the air blowing period thereof by 5 seconds from the reference value, while simultaneously increasing the air blowing period of the cold air blast applied to the chest by 5 seconds.

With the configuration described heretofore, the air volume and the air blowing period of the cold air blast applied to the face of the driver wearing contact lenses are reduced to allow a reduction in the discomfort felt by the driver wearing contact lenses who is likely to feel uncomfortable when the contact lenses are dried with the air blast. In addition, while the air blowing period of the cold air blast applied to the face is reduced, the air blowing period of the cold air blast applied to the chest is correspondingly increased so that the effect of maintaining wakefulness is likely to be maintained.

In the case of using a configuration which selects, from among the plurality of characteristic features of the driver, a predetermined number of the characteristic features in order of priority, it is appropriate to take the following procedure. When the contact lens specification portion 416 specifies the characteristic feature of the driver as wearing contact lenses and the awakening stimulation control portion 440 selects the characteristic feature of wearing contact lenses based on the priority, the awakening stimulation control portion 440 may appropriately reduce the air volume and the air blowing period of the air blast applied to the face, while increasing the air blowing period of the air blast applied to the chest.

When the awakening stimulation control portion 440 changes the temperature of the cold air blast for the region to which the cold air blast is to be applied based on the characteristic feature of the driver specified by the characteristic feature specification portion 410, it is preferable that the awakening stimulation control portion 440 also inevitably changes the air volume of the cold air blast for the region in response to the temperature change of the cold air blast. By doing so, even when the driver is less likely to sense a temperature change of the cold air blast, by changing the air volume with the same timing, the driver is more likely to sense the temperature change. As a result, the driver becomes more conscious of the effect of improving the temperature of the cold air blast, the consciousness of the effect of improving the temperature of the cold air blast further increases the improving effect, and the feeling of discomfort can further be reduced.

The awakening stimulation control portion 440 may also be configured such that, when the temperature of the cold air blast is to be changed based on the characteristic feature of the driver, the air volume of the cold air blast is inevitably changed in response to the temperature change thereof while, when the air volume of the cold air blast is to be changed based on the characteristic feature of the driver, the temperature of the cold air blast is not necessarily changed in response to the air volume change. This is because an air volume change is conceivably more likely to be sensed than a temperature change.

When increasing the air blowing period of the cold air blast applied to a given region of the driver, the awakening stimulation control portion 440 preferably correspondingly reduces the air blowing period of the cold air blast applied to another region, while increasing the air blowing period of the cold air blast applied to the given region. On the other hand, when reducing the air blowing period of the cold air blast applied to a given region of the driver, the awakening stimulation control portion 440 preferably correspondingly increases the air blowing period of the cold air blast applied to another region, while reducing the air blowing period of the cold air blast applied to the given region.

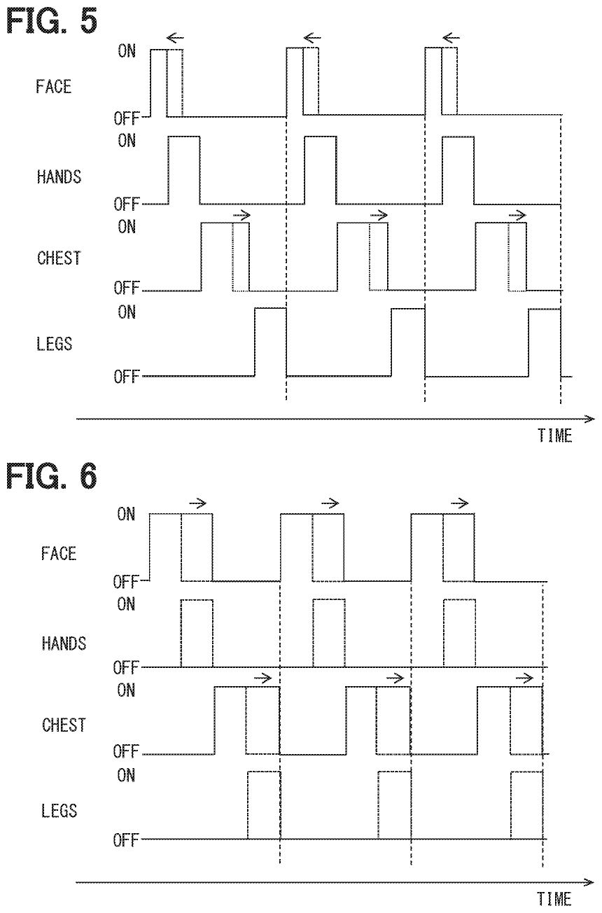

Using FIG. 5, a description is given herein of an example of changes in air blowing periods in the awakening stimulation control portion 440. FIG. 5 shows respective times at which the cold air blasts are blown toward the individual regions, i.e., the face, the hands, the chest, and the legs and respective air blowing periods of the cold air blasts. In FIG. 5, the ordinate axis of the graph represents the turning ON/OFF of the cold air blasts, while the abscissa axis represents time. FIG. 5 shows a case where the characteristic feature of the driver is female or wearing contact lenses. In FIG. 5, each of the dotted lines represents an example of a default case, while each of the solid lines represents an example of a case where the time at which the cold air blast is blown and the air blowing period of the cold air blast are changed based on the characteristic feature of the driver.

As shown in FIG. 5, when the characteristic of the driver is female or wearing contact lenses, the awakening stimulation control portion 440 reduces the air blowing period of the cold air blast applied to the face and correspondingly increases the air blowing period of the cold air blast applied to the chest, while successively changing the region to which the cold air blast is to be applied in order of the face, the hands, the chest, and the legs in rotation. As shown in FIG. 5, by reducing the air blowing period of the cold air blast applied to the face and correspondingly increasing the air blowing period of the cold air blast applied to the chest, the period of one rotation cycle is kept unchanged. Thus, while the air blowing period of the cold air blast applied to a given region is changed, the period of one rotation cycle is held constant, resulting in easier control of rotation. In addition, since the total air blowing period of the cold air blasts applied to all the regions of the driver is not reduced, the effect of maintaining wakefulness is more likely to be maintained.

Note that, in the first embodiment, the case where the characteristic features of the driver based on which the air blowing periods of the cold air blasts are to be changed are female and wearing contact lenses has been described by way of example, but the characteristic features of the driver are not limited thereto. For example, the awakening stimulation control portion 440 may also be configured such that, when the characteristic features of the driver are other than female and wearing contact lenses, the air blowing periods of the cold air blasts are changed.

When changing the region of the driver to which the cold air blast is to be applied while keeping the cold air blast from being applied to a given region thereof driver, the awakening stimulation control portion 440 preferably changes the region to which the cold air blast is to be applied to another region thereof instead of applying the cold air blast to the given region.

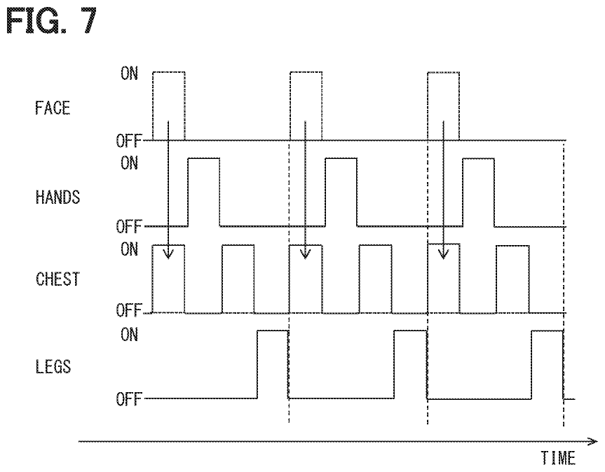

Using FIGS. 6 and 7, a description will be given of an example of changes in regions to which the cold air blasts are to be applied in the awakening stimulation control portion 440. FIGS. 6 and 7 show respective times at which the cold air blasts are blown toward the individual regions, i.e., the face, the hands, the chest, and the legs and the respective air blowing periods of the cold air blasts. In each of FIGS. 6 and 7, the ordinate axis represents turning ON/OFF of the cold air blasts, while the abscissa axis represents time. FIG. 6 shows a case where the characteristic feature of the driver is having cold intolerance. FIG. 7 shows a case where the characteristic feature of the driver is being meticulous. In FIGS. 6 and 7, each of the dotted lines represents an example of a default case, while each of the solid lines represents an example of a case where the time at which the cold air blast is blown and the air blowing period of the cold air blast are changed based on the characteristic feature of the driver.

As shown in FIG. 6, when the characteristic feature of the driver is having cold intolerance, the awakening stimulation control portion 440 successively changes the region to which the cold air blast is to be applied in order of the face, the hands, the chest, and the legs in rotation. At the same time, the awakening stimulation control portion 440 keeps the cold air blast from being applied to the hands, while applying the cold air blast to the face during a time period during which the cold air blast is supposed to be applied to the hands and keeps the cold air blast from being applied to the legs, and applies the cold air blast to the chest during a time period during which the cold air blast is supposed to be applied to the legs. As shown in FIG. 6, during the time period during which the cold air blasts are kept from being applied to the hands and the legs, the cold air blasts are applied instead to the face and the chest so that the period of one rotation cycle is kept unchanged. Thus, while the air blowing period of the cold air blast applied to a given region is changed, the period of one rotation cycle is held constant, resulting in easier control of rotation.

As shown in FIG. 7, when the characteristic feature of the driver is being meticulous, the awakening stimulation control portion 440 successively changes the region to which the cold air blast is to be applied in order of the face, the hands, the chest, and the legs in rotation. At the same time, the awakening stimulation control portion 440 keeps the cold air blast from being applied to the face, while applying the cold air blast to the chest during a time period during which the cold air blast is supposed to be applied to the face. As shown in FIG. 7, during the time period during which the cold air blast is kept from being applied to the face, the cold air blast is applied instead to the chest so that the period of one rotation cycle is kept unchanged. Thus, while the air blowing period of the cold air blast applied to a given region is changed, the period of one rotation cycle is held constant, resulting in easier control of rotation. In addition, since the total air blowing period of the cold air blasts applied to all the regions of the driver is not reduced, the effect of maintaining wakefulness is more likely to be maintained.

Note that, in the first embodiment, the case where the characteristic features of the driver based on which the regions to which the cold air blasts are to be applied are changed are having cold intolerance and being meticulous has been described by way of example, but the characteristic features of the driver are not limited thereto. For example, the awakening stimulation control portion 440 may also be configured such that, when the characteristic features of the driver are other than having cold intolerance and being meticulous, the regions to which the cold air blasts are to be applied are changed.

With respect to the region of the driver estimated as being exposed to sunlight by the sunlight-exposed-region estimation portion 420, even when the region is a target region with respect to which the air blast stimulation condition is to be changed based on a cold-related characteristic feature, such as being cold-natured or having cold intolerance, specified by the characteristic feature specification portion 410, the awakening stimulation control portion 440 does not allow the air blast stimulation condition to be changed. On the other hand, with respect to the region of the target person other than the region thereof estimated as being exposed to sunlight by the sunlight-exposed-region estimation portion 420, when the region is the target region with respect to which the air blast stimulation condition is to be changed based on the cold-related characteristic feature specified by the characteristic feature specification portion 410, the awakening stimulation control portion 440 causes the air blast stimulation condition to be changed. The awakening stimulation control portion 440 may appropriately be configured such that, with respect to the region of the driver estimated as being exposed to sunlight by the sunlight-exposed-region estimation portion 420, even when the region is the target region with respect to which the quantitative condition for weakening the cold air blast is to be changed based on the cold-related characteristic feature, the awakening stimulation control portion 440 does not allow the quantitative condition to be changed. On the other hand, with respect to the region of the target person other than the region thereof estimated as being exposed to sunlight by the sunlight-exposed-region estimation portion 420, when the region is the target region with respect to which the quantitative condition for weakening the cold air blast is to be changed based on the cold-related characteristic feature, the awakening stimulation control portion 440 causes the quantitative condition to be changed. Examples of a change in the quantitative condition for weakening the cold air blast include a change corresponding to an increase in the temperature of the cold air blast, a change corresponding to a reduction in the air volume of the cold air blast, and a change corresponding to a reduction in the air blowing period of the cold air blast.

By way of example, when the cold constitution specification portion 413 specifies that the characteristic feature of the driver is having cold intolerance, the hands and the legs are target regions from which the region to which the cold air blast is to be applied is to be changed. When the sunlight-exposed-region estimation portion 420 estimates that the hands are exposed to sunlight, but the legs are not exposed to sunlight, the awakening stimulation control portion 440 may appropriately be configured not to allow the region to which the cold air blast is to be applied to be changed from the hands to the face, but to cause the region to which the cold air blast is to be applied to be changed from the legs to the chest.

When the hot/cold-natured characteristic specification portion 412 specifies the characteristic feature of the driver as being cold-natured, the hands and the legs are the target regions with respect to which the air volumes of the cold air blasts and the temperatures of the cold air blasts are to be changed. When the sunlight-exposed-region estimation portion 420 estimates that the hands are exposed to sunlight, but the legs are not exposed to sunlight, the awakening stimulation control portion 440 may appropriately be configured not to allow the air volumes of the cold air blasts applied to the hands to be reduced and allow the temperatures of the cold air blasts applied to the hands to be increased, but to cause the air volumes of the cold air blasts applied to the legs to be reduced and cause the temperatures of the cold air blasts applied to the legs to be increased.

With the configuration described heretofore, the cold air blast applied to the region warmed by being exposed to sunlight is weakened to be able to reduce the discomfort felt by the driver due to excessive warming of the region, while the cold air blast applied to the region not warmed by being exposed to sunlight is not weakened to be able to reduce the discomfort felt by the driver due to excessive cooling of the region.

<Summary of First Embodiment>

With the configuration of the first embodiment, based on the characteristic feature of the driver specified by the characteristic feature specification portion 410, such as gender, being hot/cold-natured, having cold intolerance, being meticulous, wearing eyeglasses, or wearing contact lenses, at least any of the region of the driver to which the cold air blast generated from the air conditioning device 6 is to be applied, the air volume thereof, the temperature thereof, and the air blowing period thereof is changed. The characteristic features such as gender, being hot/cold-natured, having cold intolerance, being meticulous, wearing eyeglasses, and wearing contact lenses are presumed to be highly related to sensitivity to the cold air blast. Accordingly, the region of the driver to which the cold blast is to be applied and quantities such as the air volume of the cold air blast, the temperature thereof, and the air blowing period thereof may cause increased discomfort to the driver having such characteristic features. Since at least any of the region of the driver to which the cold blast is to be applied, the air volume of the cold air blast, the temperature thereof, and the air blowing period thereof is changed based on the characteristic feature specified by the characteristic feature specification portion 410, it is possible to change the cold air blast based on the characteristic feature of the driver, while maintaining the wakefulness of the driver using the cold air blast and reducing the probability of causing increased discomfort to the driver. As a result, it is possible to further reduce a feeling of discomfort caused to each of the target persons by the cold air blast for maintaining wakefulness.

Note that the mode in which the air blast stimulation condition is changed based on the characteristic feature of the driver, which has been described in the first embodiment, is only exemplary, and the air blast stimulation condition may also be changed in another mode. Specifically, the awakening stimulation control portion 440 may appropriately be configured such that, as the characteristic feature is more sensitive to the cold air blast, the quantitative conditions of the cold air blast applied to the region highly sensitive to the cold air blast are eased or the region to which the cold air blast is to be applied is changed to the region less sensitive to the cold air blast while, as the characteristic feature is less sensitive to the cold air blast, the quantitative conditions of the cold air blast applied to the region highly sensitive to the cold air blast are tightened. Easing the quantitative conditions of the cold air blast indicates increasing the temperature of the cold air blast, reducing the air volume thereof, and reducing the air blowing period thereof relative to respective reference values. Tightening the quantitative conditions of the cold air blast indicates reducing the temperature of the cold air blast, increasing the air volume thereof, and increasing the air blowing period thereof relative to the respective reference values.

### Second Embodiment

In the description of the embodiment given above, as the examples of the characteristic features of the driver specified by the characteristic feature specification portion 410, gender, being hot/cold-natured, having cold intolerance, being meticulous, wearing eyeglasses, and wearing contact lenses are used. However, the characteristic features of the driver specified by the characteristic feature specification portion 410 are not necessarily limited thereto. For example, the characteristic features of the driver specified by the characteristic feature specification portion 410 may also be some of gender, being hot/cold-natured, having cold intolerance, being meticulous, wearing eyeglasses, and wearing contact lenses or other characteristic features.

### Third Embodiment

In each of the embodiments described above, the configuration in which the awakening stimulation control portion 440 successively changes the region of the driver to which the cold air blast is to be applied from one region to another in rotation is shown. However, a configuration to be used is not necessarily limited thereto. For example, it may be possible to use a configuration in which a cold air blast is simultaneously applied to the plurality of regions instead of successively changing the region to which the cold air blast is to be applied from one region to another in rotation. In the case of using the configuration in which the cold air blast is simultaneously applied to the plurality of regions, it may be appropriate to use a configuration which changes the regions of the driver to which the cold air blasts are to be applied based on the characteristic feature of the driver by reducing the number of the regions to which the cold air blasts are to be applied. Alternatively, it may also be possible to use a configuration in which a cold air blast is applied only to one region instead of successively changing the region to which the cold air blast is to be applied from one region to another in rotation. In this case, it may be appropriate to change the region of the driver to which the cold air blast is to be applied based on the characteristic feature of the driver by changing the only one region to which the cold air blast is to be applied to another.

### Fourth Embodiment

The awakening stimulation control portion 440 may also be configured to change the air volume and the temperature of the cold air blast so as to cause fluctuations in the cold air blast from the air conditioning device 6. The fluctuations mentioned herein show a state in which intensities of the cold air blast such as the air volume and the temperature thereof periodically fluctuate up and down around respective reference intensities. Since the intensities of the cold air blast are varied so as to cause fluctuations in the respective intensities of the cold air blasts applied to the different regions of the driver, each of the regions is less likely to be accustomed to the cold air blast, and therefore it is possible to obtain a higher awakening effect.

### Fifth Embodiment

In the description of each of the embodiments given above, as examples of the regions to which the cold air blasts from the air conditioning device 6 are to be applied, the face, the hands, the legs, and the chest are used. However, the regions to which the cold air blasts from the air conditioning device 6 are to be applied are not limited thereto. For example, the regions to which the cold air blasts from the air conditioning device 6 are to be applied may be some of the face, the hands, the legs, and the chest or may also include a region other than the face, the hands, the legs, and the chest.

### Sixth Embodiment

In the configuration shown in each of the embodiments described above, the wakefulness maintaining system 1 is used in an automobile. However, a configuration to be used is not necessarily limited thereto. The wakefulness maintaining system 1 can be used in various movable bodies and may also be configured to be used in a vehicle other than the automobile, such as a railroad vehicle or a motorcycle. Alternatively, the wakefulness maintaining system 1 may also be configured to be used in a movable body other than a vehicle, such as an aircraft or a ship. It may also be possible to use the same configuration as that of the wakefulness maintaining system 1 in a room of a house or a facility other than a movable body. In this case, a person in the room who is intended to maintain a wakeful state corresponds to the target person.

The controllers and methods described in the present disclosure may be implemented by a special purpose computer created by configuring a memory and a processor programmed to execute one or more particular functions embodied in computer programs. Alternatively, the controllers and methods described in the present disclosure may be implemented by a special purpose computer created by configuring a processor provided by one or more special purpose hardware logic circuits. Alternatively, the controllers and methods described in the present disclosure may be implemented by one or more special purpose computers created by configuring a combination of a memory and a processor programmed to execute one or more particular functions and a processor provided by one or more hardware logic circuits. The computer programs may be stored, as instructions being executed by a computer, in a tangible non-transitory computer-readable medium.

While the present disclosure has been described with reference to embodiments thereof, it is to be understood that the disclosure is not limited to the embodiments and constructions. The present disclosure is intended to cover various modification and equivalent arrangements. In addition, while the various combinations and configurations, other combinations and configurations, including more, less or only a single element, are also within the spirit and scope of the present disclosure.

What is claimed is:

1. A wakefulness maintaining apparatus comprising:
an awakening stimulation controller that controls an awakening stimulation device to generate a cold air wind for maintaining a wakeful state of a target person; and
a characteristic feature specification unit that specifies a characteristic feature of the target person, wherein:
the awakening stimulation controller changes an air wind stimulation condition based on the characteristic feature of the target person specified by the characteristic feature specification unit; and
the air wind stimulation condition includes at least one of a region of the target person which the cold air wind generated from the awakening stimulation device hits, an air volume of the cold air wind, a temperature of the cold air wind, and an air blowing period of the cold air wind.

2. The wakefulness maintaining apparatus according to claim 1, wherein:
the awakening stimulation controller switches at least the region of the target person which the cold air wind hits in order from one region to another in rotation.

3. The wakefulness maintaining apparatus according to claim 1, wherein:
the awakening stimulation controller controls to change at least the air volume of the cold air wind and the temperature of the cold air wind; and
when the awakening stimulation controller changes the temperature of the cold air wind toward the region of the target person which the cold air wind hits, based on the characteristic feature of the target person specified by the characteristic feature specification unit, the awakening stimulation controller always changes the air volume of the cold air wind toward the region according to a change of the temperature.

4. The wakefulness maintaining apparatus according to claim 1, wherein:
the characteristic feature of the target person specified by the characteristic feature specification unit includes at least one of a gender, a heat or cold sensitive person, a cold intolerance, a meticulous person, a wearing of eyeglasses, and a wearing of contact lenses.

5. The wakefulness maintaining apparatus according to claim 4, wherein:
the characteristic feature of the target person specified by the characteristic feature specification unit includes at least the gender;
the awakening stimulation controller controls to change at least the air volume of the cold air wind and the temperature of the cold air wind; and
when the characteristic feature of the target person specified by the characteristic feature specification unit indicates a female, the awakening stimulation controller reduces the air volume of the cold air wind and increases the temperature of the cold air wind, compared with a case where the characteristic feature of the target person specified by the characteristic specification unit indicates a male.

6. The wakefulness maintaining apparatus according to claim 4, wherein:
the characteristic feature of the target person specified by the characteristic feature specification unit includes at least the wearing of the contact lenses;
the awakening stimulation controller controls to change at least the air volume of the cold air wind; and
when the characteristic feature of the target person specified by the characteristic feature specification unit indicates the wearing of the contact lenses, the awakening stimulation controller reduces the air volume of the cold air wind toward a face of the target person, compared with a case where the characteristic feature of the target person specified by the characteristic feature specification unit indicates the wearing of no contact lenses.

7. The wakefulness maintaining apparatus according to claim 4, wherein:
the characteristic feature of the target person specified by the characteristic feature specification unit includes at least the heat or cold sensitive person;
the awakening stimulation controller controls to change at least the temperature of the cold air wind;
when the characteristic feature of the target person specified by the characteristic feature specification unit indicates the hot sensitive person, the awakening stimulation controller reduces the temperature of the cold air wind, compared with a case where the characteristic feature of the target person specified by the characteristic feature specification unit indicates neither the hot sensitive person nor the cold sensitive person; and
when the characteristic feature of the target person specified by the characteristic feature specification unit indicates the cold sensitive person, the awakening stimulation controller increases the temperature of the cold air wind; compared with a case where the characteristic feature of the target person specified by the characteristic feature specification unit indicates neither the hot sensitive person nor the cold sensitive person.

8. The wakefulness maintaining apparatus according to claim 4, wherein:
the characteristic feature specified by the characteristic feature specification unit includes at least the cold intolerance;
the awakening stimulation controller controls to change at least the region of the target person which the cold air wind hits;
when the characteristic feature of the target person specified by the characteristic feature specification unit indicates no cold intolerance, the awakening stimulation controller specifies each of hands, legs, and other regions of the target person as the region which the cold air wind hits; and
when the characteristic feature of the target person specified by the characteristic feature specification unit indicates the cold intolerance, the awakening stimulation controller specifies the hands and the legs among the hands, the legs; and the other regions of the target person as the region which the cold air wind does not hit.

9. The wakefulness maintaining apparatus according to claim 4, wherein:
the characteristic feature specified by the characteristic feature specification unit includes at least the meticulous person;
the awakening stimulation controller controls to change at least the air volume of the cold air wind; and
when the characteristic feature of the target person specified by the characteristic feature specification unit indicates the meticulous person, the awakening stimulation controller reduces the air volume of the cold air wind, compared with a case where the characteristic feature of the target person specified by the characteristic feature specification unit indicates no meticulous person.

10. The wakefulness maintaining apparatus according to claim 4, further comprising:
a sunlight-exposed-region estimation unit that estimates sunlight-exposed region of the target person exposed to sunlight, wherein:
the characteristic feature of the target person specified by the characteristic feature specification unit includes a cold-related characteristic feature which is at least one of the cold intolerance and the cold sensitive person;
even when the target person matches a target for which a quantitative condition is to be changed based on the cold-related characteristic feature specified by the characteristic feature specification unit; the awakening stimulation controller does not change the quantitative condition with respect to the estimated sunlight-exposed region of the target person estimated by the sunlight-exposed-region estimation unit; and
when the target person matches the target for which the quantitative condition is to be changed based on the cold-related characteristic feature specified by the characteristic feature specification unit, the awakening stimulation controller changes the quantitative condition with respect to a region other than the estimated sunlight-exposed region of the target person estimated by the sunlight-exposed-region estimation unit.

11. The wakefulness maintaining apparatus according to claim 1, wherein:
the awakening stimulation controller changes the quantitative condition which includes at least one of the air volume of the cold air wind, the temperature of the cold air wind, and the air blowing period of the cold air wind in the air wind stimulation condition;
a reference value for the quantitative condition is preliminarily determined with respect to the characteristic feature;
a set value indicating a change amount in which the quantitative condition is to be changed is preliminarily determined with respect to the characteristic feature; and
the awakening stimulation controller changes the quantitative condition to be a value obtained by adding the set value to the reference value or subtracting the set value from the reference value, the set value corresponding to the characteristic feature of the target person specified by the characteristic feature specification unit.

12. The wakefulness maintaining apparatus according to claim 11, wherein:
the characteristic feature of the target person specified by the characteristic feature specification unit includes a plurality of characteristic feature items; and
the awakening stimulation controller changes the quantitative condition to be a value obtained by adding the set value, corresponding to each of the plurality of characteristic feature items of the target person specified by the characteristic feature specification unit, to the reference value, or by subtracting the set value, corresponding to each of the plurality of characteristic feature items of the target person specified by the characteristic feature specification unit, from the reference value.

13. The wakefulness maintaining apparatus according to claim 12, wherein:
the plurality of characteristic feature items of the target person specified by the characteristic feature specification unit has a priority preliminarily determined according to each characteristic feature item; and
the awakening stimulation controller selects a predetermined number of the characteristic feature items among the plurality of characteristic feature items of the target person specified by the characteristic feature specification unit, in order of the priority; and
the awakening stimulation controller changes the quantitative condition to be a value obtained by adding the set value, corresponding to each of selected characteristic feature items having the predetermined number of the plurality of characteristic feature items of the target person, to the reference value, or by subtracting the set value, corresponding to each of the selected characteristic feature items having the predetermined number of the plurality of characteristic feature items of the target person, from the reference value.

14. The wakefulness maintaining apparatus according to claim 13, wherein:
the plurality of characteristic feature items of the target person specified by the characteristic feature specification unit include at least a heat or cold sensitive person; and
the priority of the characteristic feature items relating to the heat or cold sensitive person is set highest.

15. The wakefulness maintaining apparatus according to claim 13, wherein:
the priority of the characteristic feature items is determined according to a season; and
the awakening stimulation controller changes the priority of the characteristic feature items according to a present season;
the awakening stimulation controller selects a predetermined number of the characteristic feature items among the plurality of characteristic feature items of the target person specified by the characteristic feature specification unit, in order of the priority; and
the awakening stimulation controller changes the quantitative condition to be a value obtained by adding the set value, corresponding to each of selected characteristic feature items having the predetermined number of the plurality of characteristic feature items of the target person, to the reference value, or by subtracting the set value, corresponding to each of the selected characteristic feature items having the predetermined number of the plurality of characteristic feature items of the target person, from the reference value.

16. The wakefulness maintaining apparatus according to claim 11, wherein:
a maximum value and a minimum value of the quantitative condition are preliminarily determined within a range narrower than a settable range of the awakening stimulation device; and
the awakening stimulation controller changes the quantitative condition within a range not more than the maximum value and not less than the minimum value.

17. The wakefulness maintaining apparatus according to claim 1, wherein:
the awakening stimulation controller changes at least the region of the target person which the cold air wind hits and the air blowing period of the cold air wind;
when the awakening stimulation controller increases the air blowing period of the cold air wind with respect to a certain region of the target person, the awakening stimulation controller reduces the air blowing period of the cold air wind with respect to another region of the target person correspondingly by an increased air blowing period of the cold air wind with respect to the certain region; and
when the awakening stimulation controller reduces the air blowing period of the cold air wind with respect to the certain region of the target person, the awakening stimulation controller increases the air blowing period of the cold air wind with respect to the another region of the target person correspondingly by a reduced air blowing period of the cold air wind with respect to the certain region.

18. The wakefulness maintaining apparatus according to claim 1, wherein:
the awakening stimulation controller changes at east the region of the target person which the cold air wind hits; and
when the awakening stimulation controller changes the region of the target person which the cold air wind hits so as not to hit the cold air wind on a certain region of the target person, the awakening stimulation controller changes the region which the cold air wind hits to another region of the target person instead of not hitting the cold air wind on the certain region.

19. A wakefulness maintaining method, comprising:
specifying a characteristic feature of a target person; and
changing at least one of a region of the target person which cold air wind hits, an air volume of the cold air wind, a temperature of the cold air wind, and an air blowing period of the cold air wind based on the specified characteristic feature of the target person, wherein:
the cold air wind is generated by an awakening stimulation device for maintaining a wakeful state of the target person.

20. A non-transitory computer readable storage medium comprising instructions being executed by a computer, the instructions causing a computer to function as:
a characteristic feature specification unit that specifies a characteristic feature of a target person; and
an awakening stimulation controller that changes at least one of a region of the target person which cold air wind hits, an air volume of the cold air wind, a temperature of the cold air wind, and an air blowing period of the cold air wind based on the specified characteristic feature of the target person, wherein:
the cold air wind is generated by an awakening stimulation device for maintaining a wakeful state of the target person.
